(19) United States
(12) Patent Application Publication
(10) Pub. No.: US 2021/0093170 A1
(43) Pub. Date: Apr. 1, 2021

(54) LIGHT GUIDE FOR DIAGNOSTIC, SURGICAL, AND/OR THERAPEUTIC DEVICE

(71) Applicant: SCHOTT AG, Mainz (DE)

(72) Inventors: Bernd Schultheis, Schwabenheim (DE); Martin Cramer, Wiesbaden (DE); Hubertus Russert, Jugenheim (DE); Holger Werner, Frankfurt (DE); Lukas Walter, Ostrich-Winkel (DE); Hildegard Voigt, Nierstein (DE); Markus Kappel, Roxheim (DE); Erwin Steiger, Saterland-Ramsloh (DE)

(73) Assignee: SCHOTT AG, Mainz (DE)

(21) Appl. No.: 17/033,162

(22) Filed: Sep. 25, 2020

(30) Foreign Application Priority Data
Sep. 26, 2019 (DE) 10 2019 125 912.6

(51) Int. Cl. A61B 1/00; A61B 1/018; A61B 1/06

(52) U.S. Cl. CPC A61B 1/00167; A61B 1/018; A61B 1/0017; A61B 1/0684; A61B 1/00103; A61B 1/063

(57) ABSTRACT

A light guide is provided that includes a fiber bundle, a jacket, a proximal end, a distal end, and a terminated end face. The fiber bundle has a plurality of optical fibers. The jacket encloses at least a part of the plurality of optical fibers and/or the fiber bundle. The jacket has a maximum outer lateral dimension that is greater than a maximum outer lateral dimension of the fiber bundle by at most 500 μm. The terminated end face is on the proximal end and/or the distal end. The terminated end face has a maximum lateral outer dimension that is not larger than the maximum outer lateral dimension of the jacket.

### CROSS REFERENCE TO RELATED APPLICATIONS

This application claims benefit under 35 USC 119 of German Application 10 2019 125 912.6 filed Sep. 26, 2019, the entire contents of which are incorporated herein by reference.

### BACKGROUND

### 1. Field of the Invention

The invention relates to a light guide for transmitting electromagnetic radiation for a diagnostic, surgical, and/or therapeutic device for being introduced into the human or animal body or for in-vitro examination of human or animal blood samples or other body cells, in particular to an endoscope or disposable endoscope, wherein the light guide has a proximal end face for incoupling and/or outcoupling of electromagnetic radiation and a distal end face for incoupling and/or outcoupling of electromagnetic radiation.

### 2. Description of Related Art

Diagnostic, surgical, and/or therapeutic devices such as endoscopes for diagnosis, for minimally invasive interventions, or for therapy are known to have rigid or flexible designs and have been sufficiently described in the literature. Nowadays, disposable endoscope are increasingly being used, in particular to increase patient safety during medical examinations, therapies and/or minimally invasive interventions, since single use allows to prevent contamination. In fact, prior art endoscopes have been designed so as to be reprocessable in terms of medical technology, i.e. they can be cleaned, sterilized and, above all, they are autoclavable.

Nevertheless, it may occasionally happen, due to incorrect application of reprocessing or unfavorable design of such devices, that the necessary reduction in the number of bacteria fails to be achieved and hence bacteria may be transferred to the patient during the next application. This can be prevented by using such disposable endoscopes.

Another aspect for the increased use of disposable endoscopes is economic assessment. In particular the reprocessing process that has to be carried out properly and regularly after each treatment implies high costs for the practicing doctor or the clinic nowadays. Moreover, high investments are required for purifying devices such as thermal disinfectors and autoclave devices and/or plasma sterilization devices, so that, overall, the use of such disposable endoscopes is justified.

Another advantage results from the fact that such disposable endoscopes can be used as transportable hand-held devices and can therefore also be employed in emergency medicine, in military emergency missions or in regions that are difficult to access, for example during disaster relief missions, where in particular reprocessing options are not available.

Such disposable endoscopes, also known as single-use endoscopes, as described in the literature have been described in the following documents, for example:

Document U.S. Pat. No. 3,581,738 A1 discloses a disposable endoscope comprising a body of synthetic resinous material having a generally tubular side wall defining a speculum and a unitary elongated light-conducting member embedded in the side wall, the member being formed of a light-conducting material clad with a transparent material having an index of refraction different from that of the light-conducting material, the body being formed of two mating halves divided axially of the endoscope, each half having a member-enclosing wall.

Document U.S. Pat. No. 4,964,710 A1 describes a rigid endoscope equipped with an objective lens system, an ocular lens and an intermediate relay lens. The relay system is a hybrid system that uses both plastic and glass components. The plastic components comprise an even number (N) of axially aligned lenses, each having a length which is of the order of their diameter. The glass components comprise an odd number (N minus 1) of axially aligned plano glass cylinders with polished end faces.

Document EP 1890173 A1 discloses a method for producing an optical light guide that can be used in such endoscopes. A plurality of optical fibers are bundled, and the fiber bundle is cut at a part of a mouthpiece which is fixed to an intermediate part of the fiber bundle. Thus, the fiber bundle is divided into a first optical fiber bundle and a second optical fiber bundle. Separation surfaces of the first and second optical fiber bundles have the same properties and condition since the first and second optical fiber bundles are formed of the fiber bundle that is obtained by bundling the same optical fibers. The first optical fiber bundle is assembled in an insertion section of an endoscope and the second optical fiber bundle is assembled in a flexible tube, whereby a first light guide is formed in the insertion section of the endoscope and a second light guide is formed in the flexible tube. Thereby, a separable light transmission path of the light guide is formed.

Since such endoscopes are subject to high cost pressure due to their single use, the assemblies and components have to be producible in a cost-effective way. Among the main components for imaging and illumination are light guides or image guides, and these are currently still assembled and processed in rather complex processing steps. What makes the current light guides or image guides comparatively expensive is often due to complex mechanical components partly combined with optical elements such as lenses that form part of such light guides or image guides, and sometimes complex processing steps such as grinding and polishing of the end faces are moreover involved.

On the other hand, particular lighting requirements must also be taken into account when using endoscopes, especially in medical technology. In addition to transmitting the light provided by a light source to the examination site in the best possible loss-free manner, this includes a true-to-color or an intentionally colored representation of the examination site and also the avoiding of introducing unnecessary heat to the examination site.

If active electronic components are used, such as camera chips and/or LEDs for lighting, it is moreover necessary to take into account requirements with regard to electrical insulation, electrical shielding and patient leakage currents, which must not exceed maximum threshold values, depending on the field of application of the endoscope. For applications at the heart, for example, a maximum leakage current of 10 μA is required, corresponding to CF classification (see EN 60601-1, 3^{rd} edition, tab. 3).

In addition to these illumination-related and electrical requirements, requirements regarding biocompatibility must also be observed. For biocompatibility it is necessary to ensure that the material is compatible with the human organism. For medical devices that might come into contact with the human body, regulatory requirements request to determine and assess possible interactions and undesirable side effects. The selection of the required tests depends on the type of contact and duration of contact in the human body. According to European Medical Device Directive MDD 93/42 EEC (MDD for short) and Regulation (EU) 2017/745 of Apr. 5, 2017 (MDR for short), this biological assessment of a product is always necessary if there is direct contact between the material or product and the patient.

The main standards for biological tests and evaluation of materials are DIN EN ISO 10993 and the test according to United States Pharmacopeia Class VI (USP Class VI). Although the much more extensive ISO 10993 was originally intended to replace the test according to USP Class VI, the USP test is used very frequently today in particular for evaluating biocompatible plastics. For this purpose, the materials intended for invasive application are evaluated with regard to their chemical compounds on the one hand, and are on the other hand subjected to a cytotoxicity test in which possible toxic effects to living cell cultures are examined. The requirements for this are summarized in DIN EN ISO 10993, especially in parts -1 and -5 (DIN EN ISO 10993-1: 2010-04). In the United States, this is subject to FDA requirements. The requirements corresponding to DIN EN ISO 10993 are specified in USP Class VI there. DIN EN ISO 10993 in particular also defines the biocompatibility of plastics in conjunction with, for example, medical and/or therapeutic applications.

Furthermore, biocompatibility is relevant in conjunction with food, feeding stuff, and consumer goods, as regulated in LFGB (German: Lebensmittel- and Futtermittelgesetzbuch; Food and Animal Feed Code) in Germany, for example, as well as in corresponding European legal standards.

Another advantage of the endoscopes in the form of single-use endoscopes is that they do not require to take into account the known reprocessing methods in the form of cleaning or disinfection processes involving strongly basic solutions and sterilization by autoclaving at temperatures of up to 135° C. and typical steam pressures of about 3 bar, which in particular permits to choose more cost-effective materials. Only RoHS (Restriction of Hazardous Substances) and REACH (Registration, Evaluation, Authorisation and Restriction of Chemicals) regulations have to be considered for the materials.

The present Applicant's own application with application number DE 10 2018 107 523 describes illumination light guides or image guides for disposable endoscopes or assemblies comprising illumination light guides, image guides, and/or cameras, which are particularly cost-effective in manufacture while meeting typical lighting requirements for endoscopes in medical technology, in particular high transmittance, and providing for good color reproduction, in combination with high biocompatibility and low cytotoxicity in compliance with the medical requirements and effects. According to the present Applicant's own application DE 10 2018 107 523 this is achieved by proximal and/or distal end faces of the illumination light guide and/or of the image guide for assemblies comprising an illumination light guide and/or an image guide and/or a camera, such as for example a disposable endoscope, which end faces comprise at least one transparent plastic element at least partially or in sections thereof, or which have a transparent plastic molded to the proximal and/or distal end faces. The transparent plastic is biocompatible and/or has non-toxic properties to human or animal cell cultures for exposure durations of less than one day. This allows to produce illumination light guides or image guides in a very cost-effective way, while allowing to dispense with otherwise complex processing of the ends (also known as termination), i.e. grinding and polishing of the proximal or distal end faces. The biocompatibility and the non-toxic properties of the plastics provide for invasive intervention in the body (in vivo) or enable in-vitro examinations on cell cultures and/or blood samples without damaging or alterating them. By appropriately choosing the plastic it is possible to provide high-quality optical systems that meet the lighting requirements for endoscopes, especially since in the case of disposable endoscopes the temperature resistance of the plastic can in particular be lower, providing for a less limited choice. Suitable plastics include polymers from at least one of the material classes of cyclo-olefin copolymers, polycarbonates, polyethylene terephthalates, perfluoroalkoxy polymers, polyvinylidene fluorides, polymethyl methacrylates, polymethyl methacrylimides, acrylic-styrene-acrylonitrile copolymers, or room temperature crosslinking silicone, hot crosslinking liquid silicones, epoxy casting resins or adhesives, thermally or UV crosslinking acrylate casting resins, polyurethane casting resins, polyester casting resins, or mixtures and/or combinations thereof. Care must be taken to choose respective biocompatible variants that meet the requirements of the standards mentioned in the introductory part. In this respect, particularly suitable materials are thermoplastics which are easily injection molded and which are transparent, e.g. PC, PMMA, COC, etc., but also plastics that can be applied as casting resin and which permit to achieve respective smooth surfaces with a very low roughness value. Moreover, the plastics mentioned above are available in a biocompatible version.

### SUMMARY

It has been found that there is a particular need for very thin light guides. This is because it is very difficult for the prior art light guides to be inserted into a diagnostic, surgical, and/or therapeutic device for being introduced into the human or animal body or for in-vitro examination of human or animal blood samples or other body cells, in particular an endoscope or a single-use endoscope. Moreover, it is disadvantageous that a large part of the end face of the light guide is reduced in size by constituents of the light guide which do not conduct light, i.e. in particular by a sheath or jacket and/or in particular by a ferrule fitted to the distal end of the light guide.

Therefore, the object of the invention is to provide a light guide and/or a diagnostic, surgical, and/or therapeutic device which at least mitigate the deficiencies hitherto existing in the prior art.

Therefore, according to one aspect, the present disclosure relates to a diagnostic, surgical, and/or therapeutic device for being introduced into the human or animal body or for in-vitro examination of human or animal blood samples or other body cells, in particular an endoscope or a disposable endoscope that comprises a light guide comprising at least two optical fibers, the at least two optical fibers defining a fiber bundle, and a jacket enclosing the fibers and/or the fiber bundle at least partially or in sections thereof, wherein the light guide has a maximum outer lateral dimension of its cross-sectional area, for example an outer diameter, which is greater than the maximum outer lateral dimension, for example the outer diameter, of the cross-sectional area of the fiber bundle by at most 500 μm, preferably by at most 2000 μm, more preferably by at most 100 μm and most preferably by not more than 50 μm, wherein the light guide has a proximal end and a distal end and has at least one terminated end face on at least one end, preferably on the distal end, wherein the terminated end face has a maximum lateral outer dimension, for example an outer diameter, which is not larger than the maximum outer lateral dimension, for example the outer diameter, of the cross-sectional area of the light guide.

Here, a jacket enclosing the fibers and/or the fiber bundle at least partially or in sections thereof is understood to mean that the jacket may optionally leave free some outer surface areas of the fiber bundle.

Another aspect of the present document relates to a light guide comprising at least two optical fibers, the at least two optical fibers defining a fiber bundle, and a jacket enclosing the fibers and/or the fiber bundle at least partially or in sections thereof, wherein the light guide has a maximum outer lateral dimension of its cross-sectional area, for example an outer diameter, which is greater than the maximum outer lateral dimension, for example the outer diameter, of the cross-sectional area of the fiber bundle by at most 500 μm, preferably by at most 200 μm, more preferably by at most 100 μm, and most preferably by not more than 50 μm, wherein the light guide has a proximal end and a distal end and has at least one terminated end face on at least one end, preferably on the distal end, wherein the terminated end face has a maximum lateral outer dimension, for example an outer diameter, which is not larger than the maximum outer lateral dimension, for example the outer diameter, of the cross-sectional area of the light guide.

Such a configuration of the diagnostic, surgical, and/or therapeutic device and of the light guide has a number of advantages.

In fact, it has been found that with the light guides of the prior art difficulties are encountered when introducing these light guides into a diagnostic, surgical, and/or therapeutic device that is intended for introduction into the human or animal body or for in-vitro examination of human or animal blood samples or other body cells, such as an endoscope. This is because the fibers of such a light guide have to be constrained, meaning they have to be provided in such a way that the bundle does not split or fan out at least at one end, preferably at the distal end, which would otherwise make it impossible to insert them into a respective diagnostic, surgical, and/or therapeutic device. Therefore, light guides usually have a ferrule at one end, preferably at the distal end, which prevents such fanout or splitting. Mostly, the fibers or the fiber bundle are usually furthermore provided with a sheath such as a shrink tube.

However, very thin light guides, that is to say light guides which only have a very small effective lateral outer dimension cannot be produced in this way. Both the sheath of the fiber bundle and the ferrule contribute to a rather large maximum outer dimension, such as an outer diameter, of the resulting light guide, and the sheath such a shrink tube and/or the ferrule have a major impact on this maximum outer dimension such as the outer diameter of the light guide. Therefore, the light guide according to the present disclosure is designed in such a way that it has a jacket which encloses at least sections of the fibers making up the light guide or the fiber bundle making up the light guide, and so that the light guide has a maximum outer lateral dimension such as an outer diameter which is larger than the maximum outer lateral dimension such as the outer diameter of the cross-sectional area of the fiber bundle by not more than 500 μm, preferably by at most 200 μm, more preferably by at most 100 μm, and most preferably by not more than 50 μm.

The light guide has a proximal end and a distal end and at least one terminated end face on at least one end, preferably on the distal end, and the terminated end face has a maximum lateral outer dimension such as an outer diameter, which is not larger than the maximum outer lateral dimension such as the outer diameter of the light guide.

In other words, the jacket and/or the terminated end face are designed such that they only slightly increase the maximum outer lateral dimension of the cross-sectional area of the light guide. The maximum outer lateral dimension of the light guide is of particular interest because it is ultimately decisive for whether or not the light guide will fit through a particular opening and is therefore suitable for being introduced into a diagnostic, surgical, and/or therapeutic device.

If the light guide has a round or circular cross-sectional area, at least within measurement accuracy, this maximum outer lateral dimension of the cross-sectional area may be the outer diameter of the light guide, for example.

### BRIEF DESCRIPTION OF THE DRAWINGS

The invention will now be explained in more detail with reference to exemplary embodiments illustrated in the figures, wherein:

FIG. 1 is a highly simplified schematic diagram of a disposable endoscope in the form of a flexible endoscope;

FIG. 2 is a highly simplified schematic diagram of a disposable endoscope in the form of a rigid endoscope;

FIG. 3 schematically illustrates the structure of a light guide;

FIG. 4 is a sectional view schematically illustrating the structure of the light guide; and

FIGS. 5 and 6 schematically show steps of a hot forming process involving individual glass optical fibers with a polymer jacket.

### DETAILED DESCRIPTION

In the context of the present disclosure, the following definitions shall apply:

A terminated end face is a closed light exit face. Terminated end face can in particular be understood to mean an end face which has an optical quality or where only low optical loss occurs. For example, only low optical loss occurs when the end face has a low surface roughness. Here, a terminated end face may have a surface roughness, Rₐ, of at most 1.0 μm or less, preferably 0.5 μm or less, and most preferably 0.1 μm or less, while a practically achievable lower limit of surface roughness Rₐ is about 100 Å. Thus, in a broader sense, a terminated end face can also be understood as an end face that has predefined end face properties such as a predefined surface roughness.

The proximal end of the light guide or of the fiber bundle is that end of the light guide which is associated with an illuminating light source. Generally, the light guide and/or the fiber bundle has a proximal end associated with a light source, and a distal end not associated with the light source but with the site to be illuminated.

In the context of the present disclosure, the terms of terminated end face and end face with termination are largely used synonymously. Unless expressly stated otherwise, “termination” is not to be understood as an item but rather describes the state of the end face. However, it is generally possible for an end face to be terminated using a separate component.

In the context of the present disclosure, a light guide is understood to mean a component designed to conduct electromagnetic radiation, in particular electromagnetic radiation in the visible spectral range, from a light source to another location so as to illuminate this other location. Therefore, a light guide in the sense of the present disclosure may in particular also refer to an illumination light guide.

Within the scope of the present disclosure, a fiber bundle comprises at least two fibers. Usually, fiber bundles comprise between 100 and 1000 fibers, depending on the exact configuration of the fiber bundle, and the exact number of fibers a fiber bundle consists of varies depending on the respective fiber diameter, with typical diameters being between 30 μm and 100 μm, and/or on the fiber bundle diameter, and the latter is in particular dependent on the exact type of the diagnostic, surgical, and/or therapeutic device, such as an endoscope. What is of particular interest within the context of the present disclosure are thin light guides and therefore also correspondingly thin fiber bundles in which the fiber bundles rather comprise about 100 fibers or a few hundred fibers.

In the context of the present disclosure, lateral dimension of an area refers to a length parameter of the area, for example the length and/or width of an area. If the area is approximately round or circular within the range of measurement accuracy, the area can be described by the diameter, for example. For a component such as a light guide or an optical fiber, several lateral dimensions may be important due to its internal structure. For example, an optical fiber comprises a core and what is known as a cladding which is arranged around the core of the fiber. Within the context of the present disclosure, a light guide may be designed so as to comprise a fiber bundle and a jacket enclosing this fiber bundle. The light guide may therefore be described by at least two lateral dimensions of a cut surface that is essentially perpendicular to the longitudinal axis of the light guide or of the fiber bundle the light guide is consisting of, and which may also be referred to as a cross section or cross-sectional area: a first lateral dimension relating to the fiber bundle, and a second lateral dimension relating to the fiber bundle and the jacket enclosing it at least partially or in sections thereof. Since the jacket is arranged around the fiber bundle, this second lateral dimension may also be referred to as the outer lateral dimension. If the light guide is approximately circular, this outer lateral dimension is also referred to as the outer diameter. If the cross-sectional area of the light guide is approximately round or circular or has a circle segment shape (e.g. semicircular), the outer diameter may also be understood to mean the maximum outer lateral dimension. In the case of a shape that is not circular or at least circle segment-shaped, for example in the case of a rectangular or at least approximately rectangular shape of the cross-sectional area, the cross-sectional area will be characterized by the maximum lateral outer dimension within the context of the present disclosure, which in the case of a rectangle may be the diagonal.

When referring to the cross-sectional area of the light guide and/or of the fiber bundle within the context of the present disclosure, this means the area that is obtained when the fiber bundle or the light guide is cut, unless expressly stated otherwise, and this section is perpendicular or substantially perpendicular to the longitudinal axis of the fiber bundle or the light guide. Here, substantially perpendicular means that the normal vector of the cross-sectional area and the longitudinal axis of the fiber bundle or of the light guide do not deviate from one another by an angle of more than 5°.

In the context of the present disclosure, jacket is understood to mean a material or a product which encloses the fiber bundle and/or the fibers the fiber bundle consists of, at least in sections thereof. The jacket may initially be formed as a separate molded part, for example, such as a tube and/or a film. However, this is not necessary, rather it is possible for the jacket to be formed in situ, for example by a fluid material enveloping and/or impregnating the fiber bundle and then being cured so as to form the jacket.

Such a configuration of the diagnostic, surgical, and/or therapeutic device or of the light guide according to the present application has a number of advantages. In particular, the light guide is made very thin and flexible in this way. By using a particularly thin jacket for the light guide, only a correspondingly small proportion of the end face, if any, will not be exploited for illumination, which results in a particularly high light intensity. In other words, this means: because of the thin jacket a maximum possible surface area of light-transmitting fibers is resulting across the cross-sectional area or end face in this way. This therefore means a maximum light transport in lumens which accordingly achieves a maximum light yield based on the available cross section for light guides according to the present disclosure in comparison to the light guides of the prior art.

According to one embodiment of the diagnostic, surgical, and/or therapeutic device and the light guide, the jacket of the light guide is made of plastic, preferably of a biocompatible plastic, in particular a biocompatible plastic in compliance with DIN EN ISO 10993 and/or a biocompatible plastic for applications in food, in particular in the sense of the German LFGB standard and/or of corresponding international and/or European legal standards, and/or a plastic that has non-toxic properties to human or animal cell cultures over exposure durations of less than one day.

In the context of the present disclosure, plastic is understood to mean a material which comprises macromolecules or is predominantly made of macromolecules, i.e. at least 50 wt % thereof, or even substantially, i.e. at least 90 wt % thereof, or even completely. In the context of the present disclosure, macromolecule refers to a molecule which has a molar mass of at least 10,000 g/mol. In the context of the present disclosure, the term plastic is used interchangeably with the term polymer, unless expressly stated otherwise.

Such a configuration of the light guide is advantageous because plastics are available in diverse variations and can be designed according to the requirements for the jacket, so that the necessary requirements for the light guide can be adapted with regard to the flexibility and/or rigidity thereof, to temperature and/or climate and/or moisture resistance, and to optical properties, for example. Due to the high flexibility in the selection of a plastic, it is also possible, for example, to use a biocompatible plastic and/or a plastic that has non-toxic properties to human or animal cell cultures over exposure durations of less than one day, and/or a sterilizable plastic, preferably one that can be sterilized using ethylene oxide.

Biocompatible material refers to a material that is compatible with the human organism within the context of the present disclosure. It can in particular be understood to mean a biocompatible material in the sense of DIN EN ISO 10993 and/or for food, consumer goods, and/or feeding stuff in the sense of the German LFGB standard and/or of corresponding international and/or European legal standards.

As already stated above, it is a regulatory requirement for medical products that might come into contact with the human body, to determine and assess possible interactions and undesirable side effects, and the selection of the required tests is based on the type of contact and the duration of contact in the human body. According to European Medical Device Directive MDD 93/42 EEC and Regulation (EU) 2017/745 (MDR), this biological assessment of a product is always necessary if there is direct contact between the material or product and the patient.

The main standards for biological tests and evaluation of materials are DIN EN ISO 10993 and the test according to United States Pharmacopeia Class VI (USP Class VI). Although the much more extensive ISO 10993 was originally intended to replace the test according to USP Class VI, the USP test is used very often today in particular for assessing biocompatible plastics. For this purpose, the materials intended for invasive application are evaluated with regard to their chemical compounds on the one hand, and are on the other hand subjected to a cytotoxicity test in which possible toxic effects to living cell cultures are examined. The requirements for this are summarized in DIN EN ISO 10993, especially in parts -1 and -5 (DIN EN ISO 10993-1: 2010-04). In the United States, this is subject to FDA requirements. The requirements corresponding to DIN EN ISO 10993 are specified in USP Class VI there.

The plastic is preferably selected from the group consisting of cyclo-olefin copolymers, polycarbonates, polyethylene terephthalates, perfluoroalkoxy polymers, polyvinylidene fluorides, polymethyl methacrylates, polymethyl methacrylimides, acrylic-styrene-acrylonitrile copolymers, or room temperature crosslinking silicone, hot crosslinking liquid silicones, epoxy casting resins or adhesives, thermally or UV crosslinking acrylate casting resins, polyurethane casting resins, polyester casting resins, or mixtures and/or combinations thereof. In particular combined crosslinking processes are also conceivable here, for example combined UV and hot crosslinking.

These materials are preferred because they are easily processible, commonly available plastics which can be produced cost-effectively in very good quality. They allow to easily produce a jacket which has good mechanical stability, so that the fiber bundle is protected against mechanical loads such as, for example, bending, but at the same time also exhibits sufficient chemical resistance.

According to a further embodiment of the diagnostic, surgical, and/or therapeutic device and/or the light guide, the jacket of the fiber bundle comprises a shrink tube, a film, an extruded jacket, and/or a material having low-viscosity flow properties, which can be applied directly to the fiber bundle by spray-coating, dip-coating, casting, and/or extrusion, essentially without an air gap, and the material the jacket is made of may be any of transparent, translucent, opaque, and/or colored. In the context of the present disclosure, an implementation according to which the jacket can be applied directly to the fiber bundle by extrusion, essentially without an air gap, is understood to mean that a possibly existing gap between the jacket or the jacket material and the fiber bundle is not larger than 10 μm, preferably significantly smaller, and most preferably no air gap at all is formed.

In particular an extrusion essentially without an air gap has hitherto not yet been implemented. In the case of what is known as extrusion without an air gap, which has been described in patent application publication DE 10 2011 114 575 A1, for example, the fiber bundle tends to break much more easily. What is known as extrusion with air gap is an extrusion in which there is no bond, in particular no material bond between the fiber bundle and the jacket. In other words, the jacket and the fiber bundle are mechanically decoupled in this case.

However, this is no longer the case in the embodiment of the diagnostic, surgical, and/or therapeutic device and/or the light guide in which the jacket can be applied directly to the fiber bundle. Rather, there is now close intimate contact between the jacket material and the material of the fiber bundle or between the jacket material and at least one fiber of the fiber bundle. Surprisingly, it has been found that in this way, contrary to what was expected, the resulting mechanical strength of the light guide is not too low. In particular, the light guide does not necessarily break more easily if the jacket is made extremely thin, for example, as described above, and/or when using particularly soft polymers (low Shore hardness, e.g. ≤50 Shore A) which are therefore flexible in their cured or crosslinked final state. The implementation of a light guide in such a way that there is close contact between the jacket and the fiber bundle or between the jacket and at least one fiber of the fiber bundle is advantageous because this allows easy and cost-effective manufacture of the light guide. More particularly, when extruding a jacket, for example, it is no longer necessary to have a fluid flow between the jacket and the surface of the fiber bundle in order to form a gap between the jacket and the surface of the fiber bundle to thereby mechanically decouple the jacket and the fiber bundle.

Preferably in this case, the jacket surrounding the fiber bundle and/or the fibers is made by extrusion, and the material has a wall thickness of not more than 0.15 mm, preferably 0.1 mm, and most preferably of not more than 0.05 mm, and the jacket material is or comprises a thermoplastic, in particular a fluorothermoplastic, and/or a high-temperature plastic, preferably a high-temperature plastic exhibiting low viscosity at the respective processing temperature, and/or a compound comprising such a thermoplastic or a high-temperature plastic.

Suitable plastic materials, especially also constituents of compounds, may in particular include:

| Plastics classes | | Exemplary |
|---|---|---|
| new/old | Designation | trade names |
| TPE-A or TPA | Thermoplastic copolyamides | PEBAX © |
| TPE-E or TPC | Thermoplastic polyester elastomers/ | Hytrel © |
| | Thermoplastic copolyesters | |
| TPE-O or TPO | Olefin-based thermoplastic | Dytron © |
| | elastomers, mainly PP/EPDM | |
| TPE-U or TPU | Urethane-based thermoplastic | Elastollan © |
| | elastomers | |
| TPE-V or TPV | Thermoplastic vulcanizates or | Santoprene © |
| | crosslinked olefin-based thermoplastic | |
| | elastomers, mainly PP/EPDM | |
| PVC | Polyvinyl chloride | Troilit © |

Furthermore, an embodiment of the diagnostic, surgical, and/or therapeutic device and of the light guide in such a way that the jacket comprises a transparent plastic can be advantageous. For example, the plastic according to this embodiment may be an optical plastic, that is to say a plastic that is used in optical applications such as for spectacle lenses and/or optical lenses, for example. This is in particular advantageous because, for example, it allows for an embodiment of the diagnostic, surgical, and/or therapeutic device and the light guide in which the fiber bundle and/or the fibers making up the fiber bundle are at least partially embedded in the jacket material and/or may be enclosed by the jacket material at least partially and/or at least in sections thereof, and furthermore this allows a design of the termination of the end face such that the end face is formed integrally with and by the jacket. Since according to one embodiment the fibers and/or the fiber bundle can be at least partially embedded in and/or enclosed by the jacket material at least partially and/or in sections thereof, this makes it possible, in the areas of the light guide where the jacket at least partially encloses the fiber bundle and/or at least the fiber, to obtain end faces, for example by cutting, at which the jacket constrains the fibers in such a way that a termination of the end face by a ferrule and/or some other separate component having no material bond to the fibers and/or the fiber bundle can be dispensed with. For example, the jacket material may generally be provided in the form of a potting compound which, emanating from the end face, penetrates into the fiber bundle at least a few millimeters, not more than ten millimeters. In this way it is particularly easy to accomplish termination of the end face such that it is formed integrally with and by the jacket. In this way it is even possible to obtain end faces which are not produced by cutting the fiber bundle, but rather are provided as a terminated end face of sufficient quality by virtue of the surface tension of the jacket material per se.

According to a further embodiment of the diagnostic, surgical, and/or therapeutic device and of the light guide, the fiber bundle comprises a glass optical fiber and/or a polymeric optical fiber (POF). In particular, this embodiment includes the case where the fiber bundle is a glass fiber bundle or a POF fiber bundle.

In the context of the present disclosure, a fiber is understood to mean a body having a largest lateral dimension in one spatial direction of a Cartesian coordinate system that is larger than in the other two spatial directions perpendicular to this first spatial direction by at least a factor of 10, preferably by at least a factor of 50. In other words, a fiber is a very long, thin body.

A glass optical fiber comprises glass. Besides the glassy material, the glass optical fiber may furthermore comprise a further material at least partially enclosing the surface of the glassy material, which is known as surface sizing. Depending on the intended use, different glassy materials can be used for a glass optical fiber. In particular, the glass fiber may comprise a one-component and/or a multi-component glass. For example, the glass optical fiber may comprise fused silica as a substantially one-component glass and/or may in particular be in the form of a fused silica optical fiber, and the fused silica may also be doped, for example doped with OH ions and/or doped with fluorine, and/or may be provided in the form of water-rich or water-poor fused silica variations, for example, which will still be referred to as a one-component glass, or may comprise a multi-component glass, for example a multi-component silicate glass. Furthermore, the glass may also be in the form of a chalcogenide glass. Fused silica optical fiber or fused silica fiber also refers to a fiber comprising doped fused silica.

The optical fiber preferably comprises a fiber core and a fiber perimeter or fiber cladding layer. In preferred embodiments, the core layer is made of a core glass. In other embodiments, the fiber core is made of a core polymer. For example, suitable core polymers include polyacrylate, polymethacrylate, polyurethane, polyester, polyamide, and mixtures thereof.

The optical fiber preferably comprises a fiber cladding surrounding the fiber core. In preferred embodiments, the fiber cladding comprises a cladding glass. In other embodiments, the fiber cladding comprises a cladding polymer. For example, suitable cladding polymers include polyacrylate, polymethacrylate, polyurethane, polyester, polyamide, and mixtures thereof.

The fiber cladding preferably has a content of halogens or halides of less than 500 ppm (m/m), more preferably less than 400 ppm (m/m), yet more preferably less than 300 ppm (m/m), yet more preferably less than 250 ppm (m/m), yet more preferably less than 200 ppm (m/m), yet more preferably less than 150 ppm (m/m), yet more preferably less than 100 ppm (m/m), yet more preferably less than 80 ppm (m/m), yet more preferably less than 60 ppm (m/m), yet more preferably less than 40 ppm (m/m), yet more preferably less than 20 ppm (m/m), most preferably less than 10 ppm (m/m). In particularly preferred embodiments, the fiber cladding is free of halogens. Halogens include chlorine, fluorine, bromine, and/or iodine, or their anions, for example. An excessive concentration of halogens in the fiber cladding will lead to a formation of the corresponding halogen acids, in particular during steam sterilization, for example. Such halogen acids may reduce the resistance of the optical fiber article and may also be released therefrom. The halogen acids in particular attack materials such as stainless steel of autoclaves and endoscopes and cause the formation of undesirable rust.

The fiber core preferably has a content of halogens or halides of less than 500 ppm (m/m), more preferably less than 400 ppm (m/m), yet more preferably less than 300 ppm (m/m), yet more preferably less than 250 ppm (m/m), yet more preferably less than 200 ppm (m/m), yet more preferably less than 150 ppm (m/m), yet more preferably less than 100 ppm (m/m), yet more preferably less than 80 ppm (m/m), yet more preferably less than 60 ppm (m/m), yet more preferably less than 40 ppm (m/m), yet more preferably less than 20 ppm (m/m), most preferably less than 10 ppm (m/m). In particularly preferred embodiments, the core layer is free of halogens. Halogens according to the invention include chlorine, fluorine, bromine, and/or iodine, or their anions, for example. An excessive concentration of halogens in the fiber core will lead to a formation of the corresponding halogen acids, in particular during steam sterilization, for example. Such halogen acids may reduce the resistance of the optical fiber article and may also be released therefrom. The halogen acids in particular attack materials such as stainless steel of autoclaves and endoscopes and cause the formation of undesirable rust.

In particular embodiments, the optical fiber is a fused silica fiber. In a particular embodiment, the fiber cladding and/or the fiber core includes a fraction of fused silica of at least 76 wt %, more preferably of at least 81 wt %, yet more preferably of at least 84 wt %, yet more preferably of at least 88 wt %, yet more preferably of at least 92 wt %, yet more preferably of at least 95 wt %, yet more preferably of at least 97 wt %, most preferably of at least 98 wt %. The higher the fraction of fused silica the better is chemical resistance and temperature resistance.

In one particular embodiment, the core glass has the following features:

The core glass preferably comprises at least 8 wt %, more preferably at least 23 wt %, yet more preferably at least 24 wt %, and most preferably at least 25 wt % or even at least 26 wt % of SiO₂. In a particular embodiment, the core glass may even comprise at least 28.3 wt % of SiO₂, most preferably at least 34 wt % of SiO₂. In some preferred embodiments, the core glass even comprises at least 35 wt % of SiO₂, more preferably at least 42 wt %.

Preferred core glasses of the present invention comprise the constituents in the composition ranges as listed below, in percent by weight:

| Component | from | to |
|---|---|---|
| B₂O₃ | 0 | 24 |
| SiO₂ | 23 | 62.1 |
| Al₂O₃ | 0 | 10 |
| Li₂O | 0 | 10 |
| Na₂O | 0 | 18.5 |
| K₂O | 0 | 25.7 |
| BaO | 0 | 57.8 |
| ZnO | 0 | 40 |
| La₂O₃ | 0 | 25 |
| ZrO₂ | 0 | 10 |
| HfO₂ | 0 | 14.2 |
| SnO₂ | >0 | 2 |
| MgO | 0 | 8 |
| CaO | 0 | 8 |
| SrO | 0 | 24.4 |
| Ta₂O₅ | 0 | 22 |
| Y₂O₃ | 0 | 11.9 |
| Rb₂O | 0 | 15 |
| CS₂O | 0 | 21 |
| GeO₂ | 0 | 7.5 |
| F | 0 | 2 |
| Σ R₂O | 5 | 20 |
| Σ MgO, CaO, SrO, | 20 | 42 |
| ZnO | | |

R₂O is the total of the respective contents of all alkali metal oxides.

One or more of the following components may be contained in the core glass: Cs₂O, Rb₂O, MgO, CaO, SrO, Gd₂O₃, Lu₂O₃, Sc₂O₃, Y₂O₃, In₂O₃, Ga₂O₃, and WO₃.

The following components should preferably not be contained in the core glass or only in concentrations of not more than 500 ppm each, such as caused by unavoidable impurities in the raw materials: TiO₂, CeO₂, Nb₂O₅, MoO₃, Bi₂O₃, PbO, CdO, Tl₂O, As₂O₃, Sb₂O₃, SO₃, SeO₂, TeO₂, BeO, radioactive elements and coloring components, unless otherwise described in the text. In particular TiO₂ should be avoided, since this component may lead to pronounced absorption in the UV range. In preferred embodiments, WO₃ is also dispensed with as a constituent.

The components TiO₂, CeO₂, Nb₂O₅, and/or Bi₂O₃ may be contained in the core glass in an amount of up to a maximum of 0.5 wt %, preferably up to 0.3 wt %, and most preferably up to 0.2 wt %. In a preferred embodiment, the core glass is free of these components.

The core glass is preferably free of optically active components, in particular Sm₂O₃, Nd₂O₃, Dy₂O₃, Pr₂O₃, Eu₂O₃, Yb₂O₃, Tb₂O₃, Er₂O₃, Tm₂O₃, and/or Ho₂O₃. CeO₂ absorbs in the UV range, so that preferred core glasses do not contain any CeO₂.

The total content of alkaline earth metal oxide components La₂O₃, Ta₂O₅, ZrO₂, and HfO₂ is preferably at least 40 wt %, more preferably at least 42 wt %, yet more preferably at least 50 wt %, and most preferably at least 55 wt %, especially for core glasses with refractive indices of greater than 1.65 wt %. If the content of these components is too low, the preferred refractive index can commonly not be obtained. Depending on the formulation, this total amount should not exceed a value of 72 wt %.

In one specific embodiment, the cladding glass has the following features: The cladding glass preferably has an SiO₂ content of >60 wt %, more preferably >65 wt %, and most preferably at least 69 wt %. The SiO₂ content is preferably not more than 75 wt % and most preferably not more than 73 wt %. The cladding glass tends to be exposed to stronger environmental impacts than the core glass. A high SiO₂ content imparts better chemical resistance. Consequently, the content of this component in the cladding glass is preferably greater than in the core glass.

The composition of the cladding glass is preferably selected or adapted to that of the core glass in such a way that the coefficient of linear thermal expansion of the cladding glass and that of the core glass differ as little as possible. Commonly, the coefficient of thermal expansion (CTE) in a temperature range from 20 to 300° C. may be the same or may be different for the fiber core and the fiber cladding. In particular, the CTE is different. Preferably, the CTE of the cladding is lower than the CTE of the fiber core, typically it is lower by at least 1.0*10⁻⁶/K, but depending on the glass it may typically also be lower by at least 2.5*10⁻⁶/K. The fiber core typically has a CTE from 6.5*10⁻⁶ to 10*10⁻⁶/K, the cladding has a CTE from 4.5*10⁻⁶ to 6*10⁻⁶/K. This ensures that the core of the fiber shrinks more than the fiber cladding upon cooling, so that a compressive stress is built up in the fiber cladding, which protects the fiber, which is beneficial for the mechanical strength of the fiber, in particular its flexural strength.

The table below shows some preferred compositions of cladding glasses that can be used in combination with the core glasses. The cladding glasses comprise (in wt % on an oxide basis):

| Oxide | Group 1 | Group 2 | Group 3 | Group 4 |
|---|---|---|---|---|
| SiO₂ | 70-78 | 63-75 | 75-85 | 62-70 |
| Al₂O₃ | 5-10 | 1-7 | 1-5 | 1-10 |
| B₂O₃ | 5-14 | 0-3 | 10-14 | >15 |
| Li₂O | none | 0-1 | 0-3 | <0.1 |
| Na₂O | 0-10 | 8-20 | 2-8 | 0-10 |
| K₂O | 0-10 | 0-6 | 0-1 | 0-10 |
| MgO | 0-1 | 0-5 | none | 0-5 |
| CaO | 0-2 | 1-9 | none | 0-5 |
| SrO | 0-1 | none | none | 0-5 |
| BaO | 0-1 | 0-5 | none | 0-5 |
| Halogen | none | none | none | none |

In another specific embodiment, the core glass and/or the cladding glass is a chalcogenide glass, which in particular allows applications in the infrared range. The table below shows preferred compositions of chalcogenide core glasses and/or chalcogenide cladding glasses, in mol percent:

| Component | Mol % |
|---|---|
| S | 50-90 |
| Ga | 0-25 |
| As | 0-40 |
| Ge | 0-35 |
| R¹ (added in the form of R¹Hal) | 0-7.25 |
| R² (added in the form of R²Hal) | 0-13.5 |
| M¹ (added in the form of M¹Hal₂) | 0-5 |
| M² (added in the form of M²Hal₂) | 0-7.25 |
| Ln (added in the form of LnHal₃) | 0-4 |
| Total of Ga, As, and Ge | 10-42 |
| Total of R¹, R², M¹, M², and Ln | 0-16 |
| Total of halogens | 0-16 |

Here, Hal=fluorine, chlorine, bromine, and/or iodine; Hal2 and/or Hal3=chlorine and/or bromine; R¹=Li, Na, K, Rb, and/or Cs; R²=Ag and/or Cu; M¹=Mg, Ca, Sr, and/or Ba; M²=Zn, Cd, Hg, and/or Pb; Ln=La, Ce, Pr, Nd, Pm, Sm Eu, Gd, Tb, Dy, Ho, Er, Tm, Ty, Lu, Y, and Sc.

It is particularly advantageous if the glass fibers, fiber rods, or pressed fiber rods are made of a core glass and cladding glass that are free of Pb and heavy metals. Such fiber systems in particular offer high transmittance in the VIS spectral range and, due to their comparatively high transmittance in the blue spectral range, exhibit high color fidelity, which is particularly important for the medical assessment of tissue. Often only slight differences in color of the tissue decide whether this is a benign or malignant tissue change. It is therefore important to have a high CRI value for the overall system comprising the light source, light guide and imaging device, with CRI (Color Rendering Index) being a key figure of a photometric parameter that describes the quality of color rendering of light sources having the same correlated color temperature. With the glass fibers, fiber rods, or pressed fiber rods described above, a CRI value of >90 can be achieved. Such fiber systems are known from the present Applicant under the name SCHOTT PURAVIS® and have been described with regard to their composition in DE 102012100233 B4 and DE 102013208838 B4. Similar fiber systems which are likewise free of Pb are furthermore described in EP 2072477 B1.

In particular for use in endoscopes it is advantageous if glass fibers, fiber rods, or pressed fiber rods are made of a glass system which has an acceptance angle 2α of greater than 80°, most preferably greater than 100° for the light to be transmitted, which corresponds to a numerical aperture (NA) of greater than 0.64, most preferably greater than 0.77. What can be achieved thereby on the one hand is that in particular light from LEDs, which usually have a very wide emission angle, can be injected into the glass fibers or fiber rods or pressed fiber rods without elevated coupling losses, and this without the need for complex optics at the proximal end. On the other hand, wide-angle illumination can be achieved on the distal end without additional optics, which is most preferably for endoscopic examinations. Optimum illumination over the currently common camera viewing angles (usually 120° diagonally) can be achieved if the glass fibers, fiber rods, or pressed fiber rods have an acceptance angle 2α of at least 120° or an NA of at least 0.86.

An embodiment of the diagnostic, surgical, and/or therapeutic device and/or of the light guide, in which the fiber bundle comprises at least one glass fiber, in particular a glass fiber comprising a multi-component silicate glass or a glass fiber made of a multi-component silicate glass, or preferably a glass fiber bundle which in particular is in the form of a glass fiber bundle comprising glass fibers comprising a multicomponent silicate glass or made of a multicomponent silicate glass or consisting of glass fibers made of a multicomponent silicate glass is particularly advantageous. This is because the optical properties of the glass fiber bundle and therefore of the light guide or of the diagnostic, surgical, and/or therapeutic device can be adapted particularly flexibly with such glass fibers. Furthermore, such light guides which are based on glass optical fibers, exhibit significantly higher temperature resistance than a polymer optical fiber (POF). This is in particular relevant if particularly good coupling efficiency is sought to be achieved and, for example, a thin fiber bundle made of or comprising glass optical fibers is directly contacted on an LED chip or arranged very close to such a chip. However, a polymer optical fiber or a fiber bundle composed of or comprising polymer optical fibers would not withstand such a thermal load, rather the fibers would melt.

According to a further preferred embodiment of the diagnostic, surgical, and/or therapeutic device or of the light guide, the light guide comprises a fiber bundle comprising a glass optical fiber which has a numerical aperture in air of greater than 0.80, preferably greater than 0.85, and in which case the core of the glass optical fiber preferably comprises a glassy material with a composition that is selected from the glass compositions and glass composition ranges for core glasses listed above. In particular, the core of the glass fiber may predominantly comprise such a glassy material, that is to say at least 50 wt %, or essentially, i.e. at least 90 wt %, or even may completely be made thereof. This is advantageous because in this way very good illumination of the camera's field of view can be achieved (here in particular for 1×1 mm² area CMOS cameras, for example).

According to a further embodiment of the diagnostic, surgical, and/or therapeutic device or of the light guide, the fiber bundle comprises at least one glass optical fiber, and the core and/or cladding glass of the glass fiber is free of lead and/or antimony and/or arsenic and/or other heavy metals, except for inevitable traces.

According to yet another embodiment of the diagnostic, surgical, and/or therapeutic device and of the light guide, at least one fiber of the fiber bundle, preferably a fiber comprising a glass or made of a glass, most preferably comprising a multicomponent silicate glass or made of a multi-component silicate glass, has a polymer layer as an outer jacket, which is preferably made of a thermoplastic material having a melting temperature range from at least 80° C. to at most 400° C., preferably at least 80° C. to at most 250° C., most preferably from at least 120° C. to 200° C. Processing temperatures from 80° C. to 400° C. may in particular be possible for fluorinated polymers or fluoropolymers. Such an embodiment is particularly advantageous in the case of designs of the light guide or of the diagnostic, surgical, and/or therapeutic device which are optimized with regard to coupling efficiency and where the proximal end of the light guide is placed very close to a light source or even comes in contact therewith. Such a light source can be an LED chip, for example, or may comprise such an LED chip.

According to a further embodiment of the light guide or of the diagnostic, surgical, and/or therapeutic device, the termination of the fiber bundle on the at least one end, preferably on the distal end of the fiber bundle, is made by applying thereto and curing a low-viscosity transparent adhesive and/or casting resin, which adhesive and/or casting resin is curable by UV light and/or thermally; and/or the termination of the fiber bundle on the at least one end of the fiber bundle, preferably on the distal end, is in a form that is made by a hot process, preferably by a hot pressing process.

According to a further embodiment of the diagnostic, surgical, and/or therapeutic device and of the light guide, the light guide has at least one of the following features: the at least one end face is provided as a ground and/or polished and/or cut, preferably a laser-cut end face; the at least one terminated end face has a surface roughness Rₐ of ≤1.0 μm, preferably ≤0.5 μm, most preferably ≤0.1 μm; the at least one terminated end face comprises a transparent plastic having a refractive index that is substantially matched to that of the core material of the fibers of the fiber bundle; the at least one terminated end face has a circular, kidney-shaped, crescent-shaped, or other irregularly shaped cross-sectional shape; the jacket material of the light guide has a low surface energy on its surface, at least in sections thereof, low surface energy being a surface energy of less than 40 mN/m, preferably less than 30 mN/m, and most preferably less than 20 mN/m; the jacket of the light guide is provided in a coated and/or pretreated form at least in sections thereof, so that the surface of the jacket material has improved sliding properties at least in sections thereof, as obtained by a physical and/or chemical pretreatment.

It is advantageous if the at least one terminated end face is provided as a ground and/or polished and/or cut, preferably laser-cut end face and/or as an end face with a surface roughness Rₐ of ≤1.0 μm, preferably ≤0.5 μm, most preferably ≤0.1 μm.

Furthermore, it may be preferable that the plastic the jacket is made of, preferably the transparent plastic of the at least one terminated end face, preferably of the terminated proximal and/or terminated distal end faces, has a typical surface roughness Rₐ of ≤1.0 μm, preferably ≤0.5 μm, most preferably ≤0.1 μm, since such embodiments of the light guide allow to minimize scattering losses on the surface, i.e. on the end face, which would otherwise lead to a reduction in the illuminance of light guides. In the case of image guides, this allows to achieve a sharp image of the illuminated object. Such low surface roughness can in particular be achieved for polished end faces. However, it may also suffice when a greater roughness is obtained—this is in particular true if a rather diffuse light distribution is desired.

According to a further embodiment of the light guide and of the diagnostic, surgical, and/or therapeutic device according to the present disclosure, the at least one terminated end face is designed so as to comprise a transparent and/or else a translucent and/or opaque and/or colored plastic which has a refractive index that is substantially matched to that of the core material of the fibers of the fiber bundle. This means that the termination of at least one end face of the light guide may thus be designed so as to at least partially comprise jacket material. In other words, this means that the jacket material forms part or may form part of the terminated end face of the light guide at least partially, and in this case the plastic comprised in the jacket and/or the jacket itself may be opaque. This means, in other words, that the plastic material that infiltrates or surrounds the fibres may be opaque, as well as the jacket or tube that surrounds the fibre bundle that has been infiltrated at least partially at or close to the end faces or at or close to at least one end face.

Such a configuration is advantageous because it allows to minimize scattering losses and the impact of scattered light on the camera, which in the case of light guides leads to an increase in illuminance and in the case of image guides suppresses artifacts caused by reflections.

A substantially identical or matched refractive index is understood to mean that the deviation between the refractive index of the fibers or fiber components and the clear transparent plastic is at most ±0.1, most preferably at most ±0.05. This already allows to achieve good results. For example, with a maximum deviation of ±0.05 the refractive indices are almost perfectly matched so that reflection losses can be neglected in the case of light guides. In the case of image guides this allows in particular to avoid ghost images as caused by multiple reflections.

The termination may have different cross-sectional shapes. Usually, the termination will be made in such a way that the terminated surface of the light guide can be described as circular, i.e. round, within the range of measurement accuracy.

Particularly preferred is an embodiment of the diagnostic, surgical, and/or therapeutic device and of the light guide in a form where the cross section of the termination has a kidney-shaped, crescent-shaped, or other irregularly shaped cross-sectional shape instead of a circular cross-section. Namely, in particular for mechanical coupling to other components of the endoscope it may be envisaged that the proximal and/or the distal end faces additionally have a respective mechanical interface in the form of a contour of the terminated end face made of plastic or injection molded to the light guide from plastic, and this plastic may differ from the transparent plastic of the proximal and/or distal end faces and/or from the plastic comprised in the jacket at least partially or in sections thereof in terms of its material, transparency, and/or color. For example, collars or shoulders and also undercut areas can be produced in this way, which allow the light guide to be coupled with a handpiece and/or a shaft of the endoscope. Also, snap-in connections may be implemented in that way, inter alia, which provide for quick assembly, which in turn contributes to reduced manufacturing costs.

If the image guide is provided in a form so that the jacket material of the light guide has a low surface energy on its surface, at least in sections thereof, and/or so that the jacket of the light guide is provided in a coated and/or pretreated form at least in sections thereof, so that the surface of the jacket material has improved sliding properties at least in sections thereof due to a physical and/or chemical pretreatment, this will be advantageous because it allows the very thin light guide according to embodiments to be more easily introduced into a diagnostic, surgical, and/or therapeutic device according to embodiments. In particular a low force will then be required for the insertion. It may in particular be advantageous if the advantageous surface properties of the jacket, i.e. the coating and/or pretreatment for achieving a low surface energy are not only provided in sections of the jacket's surface but throughout the entire surface thereof.

One option for coating and/or pretreatment at least in sections involves chemical and/or physical coating and/or activation processes, for example, by which very low surface energies of, for example, less than 40 mN/m, preferably less than 30 mN/m, and most preferably less than 20 mN/m can be achieved. For example, fluorine fumigation of the jacket is possible, in particular if the jacket is made of silicone or if the jacket comprises a silicone-containing material. But it is also possible to apply a coating comprising parylene. A coating with TopCoat, a product from Nedform BV, NL, can also be used, in particular for silicones.

According to a further embodiment of the diagnostic, surgical, and/or therapeutic device and/or of the light guide, the jacket comprises a first plastic material and a further plastic material, and/or the terminated end face comprises a first plastic material and the jacket comprises a further plastic material. In other words, the composition of the terminated end face and that of the jacket may differ in terms of the plastic material. This may in particular be advantageous if the jacket is in the form of an extruded jacket, because this is a particularly cost-effective design of a jacket and allows for many degrees of freedom with regard to the configuration of the light guide and accordingly of the diagnostic, surgical, and/or therapeutic device. According to one embodiment, the jacket comprises a further plastic material and is in the form of an extruded jacket.

Preferably, according to one embodiment, the plastic material of the extruded jacket may be a plastic that is transparent, translucent, i.e. passing light, but not clearly, opaque, i.e. not passing light, and/or colored, i.e. absorbing, at least partially or in sections thereof. In other words, it is also possible for the jacket according to this embodiment to not be entirely transparent, which may be advantageous since in this way it is possible to selectively adapt the luminous properties of the light guide. For example, a side-emitting optical fiber may be used for lateral illumination on a diagnostic, surgical, and/or therapeutic device such as an endoscope.

According to a further embodiment of the diagnostic, surgical and/or therapeutic device, the light guide comprises flexible or semi-flexible fiber bundles and the jacket is in the form of a rigid jacket at least partially or in sections thereof. In this way, a shaft for a rigid endoscope may be advantageously implemented, for example. This allows to provide flexible or rigid single-use endoscopes in a particularly cost-effective way.

According to a further embodiment of the diagnostic, surgical, and/or therapeutic device and/or of the light guide, the light guide comprises drawn and/or pressed fiber rods and is in the form of a rigid light guide. This also allows to provide in particular rigid single-use endoscopes in a particularly cost-effective way.

According to a further embodiment of the diagnostic, surgical, and/or therapeutic device and/or of the light guide, a previously extruded jacket has been divided at specific intervals or a corresponding fiber bundle section enclosed by a jacket in the form of a tube or shrink tube has been divided according to the length of the final component, and the jacket or the tube or shrink tube has been elongated relative to the fiber bundle and the resulting cavity has been filled with an optically transparent plastic such as a clear transparent and preferably self-leveling plastic, or a prefabricated clear transparent plastic part or a light guide rod or fiber rod made of glass or plastic has been inserted and fixed in the cavity. This allows to obtain respective light entry or light exit faces which can moreover form a sufficiently smooth surface, for example.

According to yet another embodiment of the diagnostic, surgical, and/or therapeutic device and/or of the light guide, the jacket section, tube or shrink tube section defining the cavity has preferably been reshaped and forms a specific light entry or light exit contour once the plastic has been cured or once the plastic part or light guide rod has been inserted.

According to a further embodiment of the diagnostic, surgical, and/or therapeutic device or of the light guide, the proximal and/or distal end faces of the light guide include further active electronic components in the form of LEDs, laser diodes, sensors, or camera chips, which can be connected to the terminated end face and/or can be fitted thereto through a snap-in connection. In this way, advantageously, a good connection is achieved between the light guide and further components of the diagnostic, surgical, and/or therapeutic device.

According to yet another embodiment of the diagnostic, surgical, and/or therapeutic device and/or of the light guide, the proximal and/or distal end faces of the light guide are in the form of an optical element for achieving specific beam shaping and have any of a planar, convex, concave surface, or a free-form surface of any desired topography.

It is particularly advantageous if the bundles or individual fibers are at least partially or section-wise enclosed by a jacket, tube, shrink tube, or netting tube or if they are protected by a shaft of the endoscope. This increases the mechanical robustness of the system.

It may be envisaged for the jacket to be made of a further plastic material and to be in the form of an extruded cable. Such cables can be produced particularly cost-effectively in a continuous process.

It is in particular possible to use inexpensive, less temperature-stable plastics for the jacket material in the aforementioned embodiment variants, since the single-use applications do not require any thermal and/or chemical preparation processes such as autoclaving (typically @ 130-140° C. in saturated water vapor) and/or thermal disinfection processes (up to 95° C., purification agent with pH 11). What is usually employed for the sterilization of disposable instruments is ethylene oxide fumigation or partly plasma-based gas sterilizations (STERAD, with hydrogen peroxide and plasma; or STERIS, only with hydrogen peroxide), which are conducted at not more than 60° C.

According to one embodiment of the diagnostic, surgical, and/or therapeutic device, it comprises electronic elements in the form of LEDs as an illumination light source. These LEDs are associated with the proximal end of the light guide, as a light source, and the proximal end of the light guide and the light source are arranged at only a small spacing from one another. This is of advantage because it provides for a particularly high coupling efficiency, which is particularly advantageously noticeable in the illuminance at the distal end of the light guide. Besides white light LEDs, RGBW LEDs may also be used as the LEDs, which permit to switch between different colors. In addition to normal examination of tissue, this moreover allows particular diagnostic examinations in which the tissue is examined under specific wavelengths. Also conceivable is a combination of white light or RGBW LEDs with LEDs that emit in the deep blue spectral range (e.g. @ 405 nm) or in the near UV range. This even allows for fluorescence excitation. With regard to heat management, it may be contemplated that the LEDs are thermally coupled to heat sinks in the handpiece of the endoscope via metallic pins.

It can be advantageous if the proximal and/or distal end faces are in the form of an optical element to achieve specific beam shaping and therefore have a planar or convex or concave surface or a free-form surface of any desired topography. For example, with suitably designed tools, the termination of the proximal end face can be formed so as to comprise condenser lenses for better injection of light, for example in order to bundle the light of the usually wide emitting LEDs for incoupling it into the fibers according to the numerical aperture thereof (between 0.55 and 0.70; e.g. SCHOTT PURAVIS® GOF70 having a numerical aperture of 0.57, SCHOTT PURAVIS® GOF85 having a numerical aperture of 0.68). A respective convex lens formed on the distal end may likewise be used advantageously, for example in order to provide imaging optics for the camera chip. Furthermore, optical elements formed in this way on the distal end of the light guide can provide for a wide-angle emission characteristic such as a spherical or ring-shaped emission characteristic. A spherical emission characteristic allows for homogeneous illumination of body cavities, for example.

All of the examples given above are suitable for providing respective inexpensive fiber-optic components or assemblies that can be installed in flexible or rigid disposable endoscopes. Here, the umbrella term ‘disposable endoscopes’ is meant to encompasses any medical devices that can be used to direct light into the interior of the body on the one hand and on the other hand to output image information to the surgeon via optics, image guides, or camera chips. This includes angioscopes for vascular examinations with flexible endoscopes, laparoscopes for examinations in the abdominal cavity, and arthroscopes for examinations of joints with rigid endoscopes, as well as ear endoscopes, rhino endoscopes, sinuscopes, or nasopharyngoscopes, each one with a rigid endoscope, for ENT examinations.

In this case, the embodiment variants of the light guides as described above can be integrated in a handpiece of the endoscope and may in part directly define a flexible portion or a shaft of the endoscope, depending on the design of the endoscope. With the elimination of sometimes very complex grinding and polishing processes and due to the simplified assembly, costs can be saved.

A further application option particularly for the light guides as described above in the various embodiment variants, besides their use in the field of medical devices, is their use for in-vitro diagnostic devices. For this purpose, such light guides may also be used as detector light guides. For example, a large number of such illumination or detector light guides are often used in a single device for parallel tests on blood samples, for example. In particular the cost advantages should be mentioned here, be it as a result of a reduction in assembly costs or due to the integration of additional functions. A biocompatible version of the plastics can be directly exploited in this case, for example in order to bring blood samples or cell cultures into direct contact with the illumination or detector light guides. Furthermore, the glass or fused silica optical fibers described above enable spectroscopic examinations and/or examinations using fluorescence excitation, due to their advantages in optical transmission.

Such light guides as described above in their different embodiments can also be used in industrial and/or consumer products beyond a medical technology environment, where the goal is in particular to provide cost-effective light guides which moreover need to have optimal optical properties. The following application examples should be mentioned here, inter alia: illumination light guides and light guides in household appliances (cooktops, dishwashing machines, refrigerators, freezer cabinets, cooking ovens, etc.) or in small kitchen appliances (blenders, toasters, table-top cooking devices, coffee machines, etc.), for example for indicating operating conditions and/or for illuminating cooking chambers or interiors, especially if they come into contact with food; home ambiance lighting; exterior/interior automotive lighting, machine lighting.

The invention will now be explained in more detail by way of examples.

### EXAMPLE 1

According to one embodiment of the diagnostic, surgical, and/or therapeutic device or of the light guide, the light guide comprises at least two optical fibers, the optical fibers defining a fiber bundle. The fiber bundle has a diameter between at least approx. 0.4 mm and at most approx. 2.0 mm in diameter. This refers to the outer diameter of the fiber bundle.

According to this embodiment, the light guide comprises a jacket in the form of a very thin shrink tube with a wall thickness that is typically in a range of at least approximately 5 μm and at most approximately 30 μm. This jacket or shrink tube may be both transparent and opaque in this case. According to the present embodiment, the jacket is formed so as to completely enclose the fiber bundle over the entire length thereof. However, more generally, without being limited to the example specifically illustrated here, it is possible for the jacket to enclose the fiber bundle only in sections thereof.

The light guide has a proximal end and a distal end, and proximal end is generally understood as the end of the light guide which is associated with a light source here, for example connected to a light source in a form-fitting manner. More generally, without being limited to the example of an embodiment of the light guide and/or of the diagnostic, surgical, and/or therapeutic device described herein, both the light guide and the fiber bundle have a proximal end, that is associated with a light source or illumination light source, and a distal end, namely the end of the light guide or of the fiber bundle which is not associated with the light source but with the site to be illuminated.

At least one of the end faces of the light guide is terminated in this case, preferably the distal end is terminated here, and in the present case the termination is made such that the fiber bundle is preferably at least partially bonded by a low-viscosity transparent adhesive or casting resin within the shrink tube.

For example it is possible that the transparent adhesive or casting resin is an epoxy casting resin or adhesive or a silicone crosslinking at room temperature or a hot crosslinking liquid silicone or a thermal or UV crosslinking acrylate casting resin or a polyurethane casting resin or a polyester casting resin or is made from a mixture and/or from combinations of these materials or comprises such materials and/or mixtures thereof.

The transparent adhesive may in particular be in the form of a UV adhesive, that is an adhesive can be cured by UV radiation. The advantage hereof is that long adhesive curing times can be avoided.

According to the present embodiment, it is possible for the jacket to be extruded around the fiber bundle in the form of a shrink tube and then shrunk onto the fiber bundle.

### EXAMPLE 2

According to a further embodiment of the diagnostic, surgical, and/or therapeutic device or of the light guide, the light guide is designed so as to comprise at least two optical fibers, the at least two optical fibers defining a fiber bundle. According to this embodiment, the jacket is made here by first impregnating and/or wetting the fiber bundle with a transparent or opaque soft resin. It is possible that the resin wets the fiber bundle only superficially in the mold, so that only the outer surface of those fibers of the fiber bundle which are arranged on the perimeter of the fiber bundle are wetted by the soft resin, for example. However, it is also possible that the resin not only wets near the surface. It is also possible, for example, that the fiber bundle is wetted to a greater extent, in particular that all fibers of the fiber bundle are wetted by the soft resin, for example. Any intermediate degrees are possible here, for example so that the wetting of the fiber bundle is stronger on the outer surface, that is to say along the perimeter of the fiber bundle, and decreases towards the center of the fiber bundle.

The resin preferably has a low Shore hardness, in particular a Shore A hardness of less than 50 or even less than 40.

The resin may be applied by spray-coating, dip-coating, or casting, for example. Thin extrusion of the material around the fiber bundle without thereby creating an air gap is also possible.

More generally, without being limited to the example specifically described herein, it is particularly preferred for the resin to be a transparent resin and/or that the resin in its solid state has a refractive index which is preferably smaller than the refractive index of the cladding.

For example it is possible according to this example, that the fiber bundle has a diameter of 0.4 mm and that a jacket with a thickness of 25 μm is used, or a diameter of 1 mm with a jacket having a thickness of 50 μm, or a fiber bundle is provided with a diameter of 2 mm and a jacket with a thickness of 250 μm, or else a fiber bundle with a thickness of 2 mm is provided with a jacket having a thickness of 25 μm, and a fiber bundle with a thickness of 0.4 mm with a jacket having a thickness of 250 μm.

According to yet another embodiment, an even thicker jacket with a thickness of 500 μm could be used.

Thus, according to one embodiment, a resulting light guide has an area ratio of the cross-sectional area of the light guide to the cross-sectional area of the fiber bundle between 1.025 and 2.64. More generally, the area ratio mentioned above can be in a range from 1.01 to 3.0 according to one embodiment. According to yet another embodiment, the area ratio can characterize the light guide irrespectively of the thickness of the jacket. For this purpose, overall, a light guide is provided which comprises at least two optical fibers, the at least two optical fibers defining a fiber bundle, and a jacket enclosing the fibers and/or the fiber bundle at least partially or in sections thereof, with an area ratio of the cross-sectional area of the light guide to the cross-sectional area of the fiber bundle in a range from 1.01 to 3.0, preferably in a range from 1.025 and 2.64, the light guide having a proximal end and a distal end and at least one terminated end face on at least one end, the terminated end face having a maximum lateral outer dimension, for example an outer diameter, which is not larger than the maximum lateral outer dimension, for example the outer diameter, of the cross-sectional area of the light guide.

### EXAMPLE 3

According to yet another embodiment, it is possible for a light guide which has a jacket formed according to Example 2 to be combined with an end face termination according to Example 1, i.e. an end face termination obtained using an adhesive, preferably at the distal end of the light guide.

Preferably in this embodiment, a UV curing adhesive and/or a UV curing casting resin can be used.

For example, it is possible in this case for a fiber bundle of an embodiment according to Example 2 to be pressed into a shape near the end face, for example by pressing in or between molds. Then a UV-curing adhesive and/or a UV-curing casting resin can be poured onto the end face from above. Selective UV exposure allows to selectively define the penetration depth of the adhesive or casting resin and thus the length of stiffening of the fiber bundle. The penetration depth may specifically be several millimeters, in particular up to a maximum of 10 mm.

Such an embodiment is moreover particularly advantageous because it allows to produce non-circular end face geometries such as crescent-shaped or kidney-shaped geometries.

Furthermore, it is generally also possible to use a thermally curing adhesive and/or a casting resin. In this case it is likewise possible to produce a specific end face geometry and also to adjust the penetration depth and thus the length of stiffening of the light guide when using such materials.

Without being limited to the exemplary embodiment described herein, it is generally possible and may even be preferred for the adhesive or casting resin to be selected such that a desired refractive index is achieved with this adhesive or casting resin. This allows to adjust the refractive indices of the end face termination and/or the jacket and/or the optical fibers relative to one another, i.e. to match the refractive indices.

Without being limited to the exemplary embodiment described herein, it is generally possible and may even be preferred for the adhesive or casting resin that the adhesive and/or the casting resin is formed into a shape of an end face termination, and that the cured adhesive and/or casting resin has the shape of an optical element such as a lens.

An embodiment according to any one of the described Examples 2 or 3 is moreover particularly advantageous because the use of a thermoplastic adhesive and/or casting resin allows for direct hot reshaping.

Furthermore, such end face termination generally allows to obtain ends that can optionally be reprocessed.

### EXAMPLE 4

According to a further embodiment of the diagnostic, surgical, and/or therapeutic device or of the light guide, the light guide comprises optical fibers made of an optical glass, i.e., generally speaking, glass optical fibers, and these glass optical fibers comprise a core glass and a cladding glass and are provided with a thermoplastic polymer layer on their outer surface at least in sections thereof, which polymer material may be applied to the fiber by dip-coating, spray-coating or extrusion without an air gap, for example.

Such a fiber bundle can particularly advantageously be combined with an end face termination as described in Examples 2 and 3 of the present disclosure by way of example. It is in particular possible to choose the processing temperatures of the plastics of the polymer layer and of the plastics that are used for the end face termination such that the end face termination can be joined to the fiber bundle through a material bond, for example, and/or such that the fiber bundle and accordingly the resulting light guide are obtained in a specific shape, especially a non-circular shape.

Generally, it is also possible for such an embodiment to choose fibers which only comprise a core glass. In this case, the cladding, i.e. the fiber cladding material, may be made of a polymer. In this case, the polymer cladding will have respective optical properties, in particular in terms of its refractive index, as is the case with conventional core-cladding fiber configurations of glass optical fibers. In particular, the refractive index of the polymer will be lower than that of the core glass in this case.

### EXAMPLE 5

It is furthermore also possible, according to another embodiment of the diagnostic, surgical, and/or therapeutic device or of the light guide, to wrap or bandage the fiber bundle with a thin film strip or a thin film. Such an approach is described in patent document DE 1 596 485 B1, for example. Wrapping is also described in DE 10 2006 040 214 B4, however for hollow fibers for separation processes.

The film or film strip preferably comprises polyimide or PET. Furthermore, the film strip or film should have a thickness of less than 30 μm, preferably even less than 10 μm.

For increased protection against dirt and moisture of the underlying fiber or fiber bundle, the film or film strip may moreover be applied with an overlap, preferably an overlap of 50% of the width of the film. In this case, the film material may be designed so as to self-adhere to the overlapping film layers (similar to cling film) by virtue of its material properties. Alternatively, it is also possible to use film materials that bond to one another in a self-fusing way or due to additional thermal or lighting process steps such as sintering in the case of film materials such as PTFE. Braiding and enmeshing processes may be carried out using one or more filament strands, and these strands may in turn consist of several different materials. Typical materials are threads made of fiberglass, glass silk, aramid, such as available under the trade name Kevlar®, carbon fibers, polyester, polyamide, such as available under the trade name Nylon®, polyamide, PTFE, such as available under the trade name Teflon®, stainless steel, gold. Thread thicknesses are in a range of <50 μm, typically ranging from 20 μm to 30 μm.

The braiding may be implemented as a single or multiple braid, e.g. crisscross braid. This braid may be placed around the core loosely or tightly.

Here, again, a respective termination of the end face can then be achieve as described in Examples 2 and 3.

However, it is also possible and may even be preferred that the end face termination is accomplished in a way so that the fiber bundle and the jacket enclosing it are terminated by laser cutting at least in sections thereof. The light guide according to embodiments of the present disclosure will have a laser-cut end face in this case.

### EXAMPLE 6

It is furthermore also possible, according to a further embodiment of the diagnostic, surgical, and/or therapeutic device or of the light guide, that the bundle has a thickness of 0.5 mm, while the jacket has a wall thickness of 0.1 mm and is made of or comprises perfluoroethylene propylene (FEP), so that a light guide with a maximum outer lateral dimension of 0.7 mm is resulting. The jacket is made by extrusion in this case. With such a thin-walled extrusion of the jacket material, a particularly flexible light guide is obtained, in particular in comparison with an embodiment of the jacket in the form of a wrapped around film. More generally, without being limited to the specific example of a light guide illustrated here, typical wall thicknesses or thicknesses of extruded jackets in particular range from 0.05 mm to 0.1 mm.

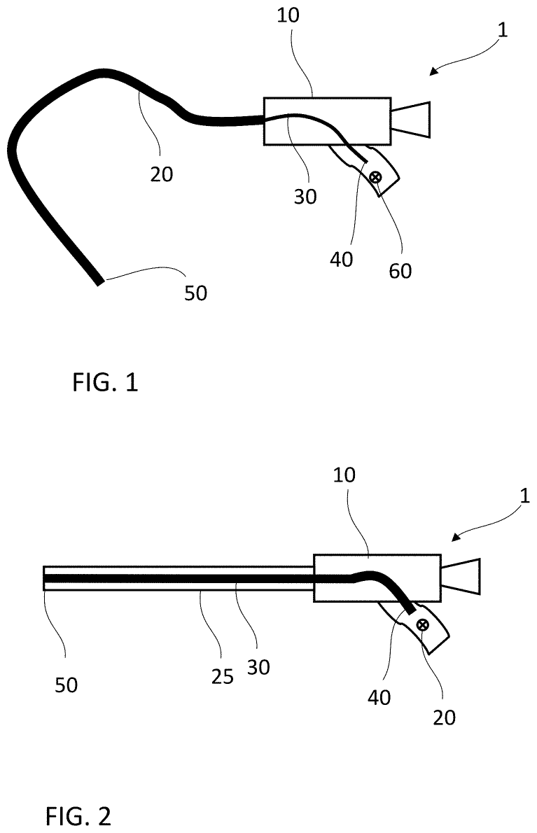

FIG. 1 schematically shows the configuration of an endoscope 1 according to the present disclosure. As an example of a diagnostic, surgical, and/or therapeutic device, a simple flexible endoscope 1 is shown here in a highly simplified manner, which comprises a handpiece 10 and a flexible section 20, the flexible section 20 being insertable into a body cavity, for example. A light guide 30 is schematically illustrated here, which has a proximal end 40 and a proximal end face 30.2 close to a lighting device in the form of an LED 60 inside the handpiece 10, and a distal end face 30.1 and a distal end 50 at the end of flexible section 20. The light from LED 60 is injected into proximal end face 30.2, i.e. at proximal end 40, and is conducted through the light guide 30 to the distal end face 30.1, i.e. distal end 50, and can then be emitted into the interior of the body through appropriate outcoupling optics. FIG. 1 does not shown the imaging components, which may include CMOS cameras, for example, which are arranged close to the distal end 50 and which electrically transmit the image information to a monitor, not shown either. Another option are fiber-optic image guides that transmit the image information to a camera or directly to an eyepiece lens. Such image guides consist of several thousands of fine individual glass fibers only a few microns in thickness, which transmit the image information pixel by pixel.

Depending on the type and application of the endoscope, the following typical dimensions are conceivable for such light guides: length between 100 mm and 3000 mm, typically 500 to 1000 mm, light guide diameter between 0.5 mm and 5 mm, typically between 1 and 2 mm.

FIG. 2 schematically shows an endoscope 1 in the form of a rigid endoscope 1, again in a highly simplified manner. Light guide 30 is routed inside a rigid shaft a25 here. The imaging or image-transmitting components as mentioned above are again not shown here, for the sake of clarity.

The exemplary embodiments or manufacturing methods that will in particular be described below mainly relate to light guides 30, but can generally be applied for image guides as well.

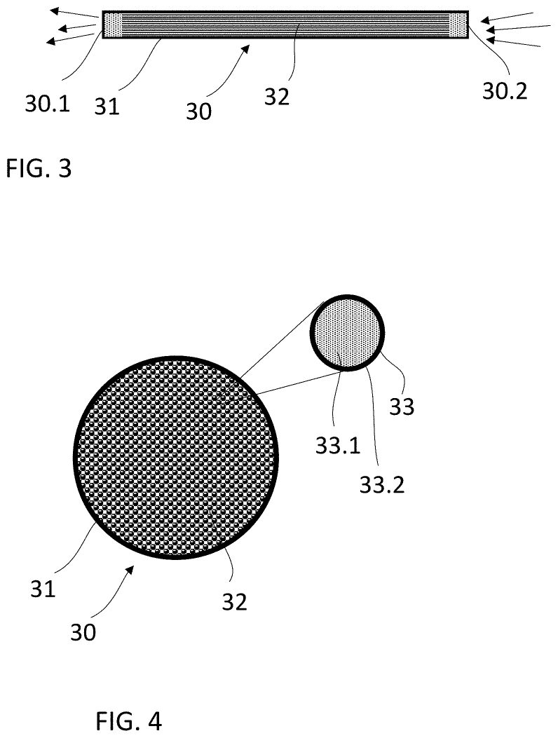

FIG. 3 is a schematic diagram of a light guide 30 according to an embodiment, not drawn to scale. Light guide 30 has a distal end face 30.1, here on the left, and a proximal end face 30.2, here on the right. Furthermore, the light guide 30 comprises a jacket 31 and at least two fibers 33 (not designated here) which form the fiber bundle 32. In other words, FIG. 3 illustrates the light guide 30 comprising at least two optical fibers 33 (not designated here), the at least two optical fibers 33 defining a fiber bundle 32, and jacket 31 enclosing the fibers 33 and/or the fiber bundle 32 at least partially or in sections thereof, and the light guide 30 has a maximum outer lateral dimension of its cross-sectional area, for example an outer diameter, which is greater than the maximum outer lateral dimension, for example the outer diameter, of the cross-sectional area of the fiber bundle by at most 500 μm, preferably by at most 200 μm, more preferably by at most 100 μm, and most preferably by not more than 50 μm; wherein the light guide 30 has a proximal end and a distal end and at least one terminated end face 30.2, 30.1 on at least one end, preferably at the distal end; wherein the terminated end face 30.2, 30.1 has a maximum lateral outer dimension, for example an outer diameter, which is not larger than the maximum outer lateral dimension, for example the outer diameter, of the cross-sectional area of the light guide 30.

FIG. 4 shows a further schematic, sectional view of a light guide 30, not drawn to scale. Here, the cross-sectional area of light guide 30 is shown, this cross-sectional area being perpendicular to the longitudinal axis of the light guide 30 or of the fiber bundle 32 or to the longitudinal axis of a fiber 33. The jacket 31 is made thin here and encloses the fibers of the fiber bundle 32, i.e. the fiber bundle 32 at least in sections thereof. Furthermore, FIG. 4 shows a schematic view of one fiber 33 which forms part of the fiber bundle 32. Fiber 33 comprises a fiber core 33.1 and a fiber cladding 33.2.

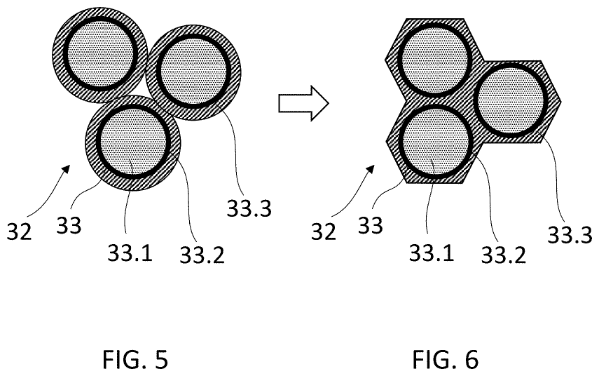

FIGS. 5 and 6 schematically illustrate a manufacturing process for a fiber bundle 32. In FIG. 5, three fibers 33 can be seen forming a fiber bundle 32. Fibers 33 each have a fiber core 33.1 and a fiber cladding 33.2 and furthermore a polymer jacket 33.3. This polymer jacket is reshaped here in a hot pressing process so as to forms a jacket 31 (not designated), as shown in the diagram of FIG. 6. In other words, in FIG. 6 the jacket is formed so as to enclose the fibers 33 of the fiber bundle and so as to not only be arranged around the perimeter of the fiber bundle 32.

### LIST OF REFERENCE NUMERALS

1 Diagnostic, surgical, and/or therapy device, here: endoscope
10 Handpiece
20 Flexible section
25 Shaft
30 Light guide
30.1 Distal end face
30.2 Proximal end face
31 Jacket
32 Fiber bundle
33 Fiber
33.1 Fiber core
33.2 Fiber cladding
33.3 Polymer jacket
40 Proximal end of light guide 30
50 Distal end of light guide 30

What is claimed is:

1. A light guide comprising:
a fiber bundle having a plurality of optical fibers;
a jacket enclosing at least a part of the plurality of optical fibers and/or the fiber bundle, wherein the jacket has a maximum outer lateral dimension that is greater than a maximum outer lateral dimension of the fiber bundle by at most 500 μm;
a proximal end;
a distal end; and
a terminated end face on the proximal end and/or the distal end, wherein the terminated end face has a maximum lateral outer dimension that is not larger than the maximum outer lateral dimension of the jacket.

2. The light guide of claim 1, wherein the maximum lateral outer dimension of the jacket and/or the fiber bundle is a diameter.

3. The light guide of claim 1, wherein the jacket comprises a material selected from a group consisting of: plastic; biocompatible plastic; sterilizable plastic; ethylene oxide sterilizable plastic; toxic plastic that is non-toxic to human or animal cell cultures over exposure durations of less than one day; cyclo-olefin copolymers; polycarbonates; polyethylene terephthalates; perfluoroalkoxy polymers; polyvinylidene fluorides; polymethyl methacrylates; polymethyl methacrylimides; acrylic-styrene-acrylonitrile copolymers; room temperature crosslinking silicone; hot crosslinking liquid silicone; UV crosslinking silicone; epoxy casting resins; epoxy casting adhesives; thermally crosslinking acrylate casting resins; UV crosslinking acrylate casting resins; polyurethane casting resins; polyester casting resins; thermoplastic, fluorothermoplastic; and any mixtures or combinations thereof.

4. The light guide of claim 1, wherein the jacket is selected from a group consisting of: a shrink tube; a film; an extruded jacket; and a material having low-viscosity flow properties.

5. The light guide of claim 1, wherein the jacket is selected from a group consisting of: a spray-coated jacket; a dip-coated jacket; a cast jacket; an extruded jacket; a transparent jacket; a translucent jacket; an opaque jacket; a colored jacket; and any combinations thereof.

6. The light guide of claim 1, further comprising no air gap between the jacket and the fiber bundle.

7. The light guide of claim 1, wherein the jacket has a wall thickness of not more than 0.15 mm.

8. The light guide of claim 1, wherein the fiber bundle comprises a glass optical fiber and/or a polymeric optical fiber.

9. The light guide of claim 1, wherein the fiber bundle comprises a glass fiber comprising or consisting of multicomponent silicate glass.

10. The light guide of claim 1, wherein the fiber bundle comprising a glass optical fiber with a numerical aperture in air of greater than 0.80.

11. The light guide of claim 1, wherein the fiber bundle comprises at least one glass optical fiber having a core and a cladding glass, wherein the core and/or cladding glass is free, except for inevitable traces, of a material selected from a group consisting of lead, antimony, arsenic, heavy metals, and any combinations thereof.

12. The light guide of claim 1, wherein at least one of the plurality of optical fibers has a polymer layer as an outer jacket made of a thermoplastic material with a melting temperature in a range between at least 80° C. and at most 250° C.

13. The light guide of claim 1, wherein the termination end face comprises a feature selected from a group consisting of: a UV cured low-viscosity transparent adhesive thereon; a UV cured low-viscosity transparent casting resin thereon; a hot-pressed end face; a ground end face; a polished end face; a laser cut end face; an end face with a surface roughness Ra of ≤1.0 μm; an end face with a transparent plastic having a refractive index substantially matched to that of the plurality of fibers; an end face with an irregularly shaped cross-sectional shape; an end face with a circular cross-sectional shape; an end face with a kidney-shaped cross-sectional shape; an end face with a crescent-shaped cross-sectional shape; a material of the jacket having surface energy of less than 40 mN/m; a material of the jacket having surface energy of less than 30 mN/m; a material of the jacket having surface energy of less than 20 mN/m; a jacket having a coating; a jacket having a physical pretreatment; and a jacket having a chemical pretreatment.

14. The light guide of claim 1, wherein the fiber bundle is flexible or semi-flexible in the jacket that is a rigid jacket or wherein the fiber bundle comprises a drawn fiber bundle that is rigid.

15. The light guide of claim 1, wherein the jacket is elongated relative to the fiber bundle so that a cavity is defined at the proximal end and/or the distal end, wherein the cavity is filled an optically transparent part.

16. The light guide according to claim 15, wherein the optically transparent part forms a light entry or light exit contour.

17. The light guide of claim 1, further comprising an active electronic component secured to the proximal and/or distal end face, wherein the active electronic component is selected from a group consisting of an LED, a laser diode, a sensors, and a camera chip.

18. The light guide of claim 1, wherein the proximal and/or distal end faces form an optical element, wherein the optical element has a shape selected from a group consisting of a planar shape, a convex shape, a concave surface shape, and a free-form surface.

19. The light guide of claim 1, further comprising an area ratio of a cross-sectional area of the light guide to a cross-sectional area of the fiber bundle of at least 1.025 and at most 2.64.

20. The light guide of claim 1, wherein the light guide is configured for a use selected from a group consisting of a diagnostic device, a surgical device, a therapeutic device, a flexible disposable endoscope, a rigid disposable endoscope, an in-vitro diagnostic device, an illumination light guide, cooktop light guide, a dishwashing machine light guide, a refrigerator light guide, a freezer cabinet light guide, a cooking oven light guide, blender light guide, a toaster light guide, a table-top cooking device light guide, a coffee machine light guide, cooking chamber light guide, a home ambiance lighting light guide, an exterior automotive lighting light guide, an interior automotive lighting light guide, and a machine lighting light guide.
